(19) United States
(12) Patent Application Publication
(10) Pub. No.: US 2009/0203586 A1
(43) Pub. Date: Aug. 13, 2009

(54) METHOD FOR AMELIORATING AN INFLAMMATORY SKIN CONDITION

(75) Inventors: Rebecca Jane Dearman, Alderley Park (GB); Marie Cumberbatch, Alderley Park (GB); Ian Kimber, Alderley Park (GB); Gregorio Del Val, Bracknell (GB)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA LIMITED, Surrey, Guildford (GB)

(21) Appl. No.: 11/570,148

(22) PCT Filed: Jun. 10, 2005
(86) PCT No.: PCT/GB2005/002300

§ 371 Date: Sep. 25, 2008

(30) Foreign Application Priority Data
Jun. 11, 2004 (GB) 0413114.0
Mar. 3, 2005 (GB) 0504426.8

(51) Int. Cl. A61K 38/17; C07K 14/00

(52) U.S. Cl. 514/12; 530/350

(57) ABSTRACT

The present invention relates to the use of thioredoxin in the manufacture of a medicament suitable for application to a skin surface for ameliorating an inflammatory skin condition. The present invention further relates to a method of ameliorating an inflammatory skin condition comprising applying to a skin surface an effective amount of a composition comprising thioredoxin. The invention further relates to a pharmaceutical composition suitable for ameliorating an inflammatory skin condition comprising from 0.0001 to 0.5 w/v thioredoxin.

The present invention relates, inter alia, to a method of ameliorating an inflammatory skin condition.

Inflammatory skin conditions are known to be associated with chemokines and cytokines, and in particular the activities of pro-inflammatory cytokines such as IL-1α, IL-1β and tumour necrosis factor a (TNF-α). These same cytokines are known also to play pivotal roles in the initiation of skin immune responses, and in fact provide mandatory signals for the migration of epidermal Langerhans cells (LC) from the skin. The movement of LC from the skin, and their subsequent accumulation in skin-draining lymph nodes provides a mechanism for the transport of antigen to the sites (regional lymph nodes) where immune responses are induced.

Our understanding that the migration of LC from the epidermis is dependent upon the provision of signals by IL-1α, IL-1β and TNF-α provides an experimental system for investigating the availability and functional activity of these cytokines in skin tissues. Experience has shown that factors that are known to inhibit the availability or function of IL-1α, IL-1β or TNF-α are associated with a significant inhibition of induced LC migration.

In addition to being required for the stimulation of LC mobilisation, IL-1β is known to cause skin inflammation and has been implicated, directly or indirectly, in the pathogenesis of several cutaneous inflammatory disorders. IL-1β is synthesised as an inactive intracellular precursor protein, which is cleaved and secreted to yield mature carboxy-terminal fragments that are biologically active and exert their effect by binding to specific cell surface receptors found on almost all cell types and triggering a range of responses.

The present invention is based on the surprising discovery that certain molecules are able, when applied topically to the skin, to inhibit the production and/or availability of bioactive IL-1α and/or IL-1β. As such these molecules are suitable, inter alia, for the treatment of inflammatory skin conditions where IL-1α and/or IL-1β are implicated in the pathogenesis. Suitable molecules include thioredoxin (TRX), a 12-kDa protein with a Cys-Gly-Pro-Cys active site, and additionally “redox-inactive” TRX molecules, wherein the cysteines at the active site are replaced by amino acids other than cysteine. Whilst it has been shown that these molecules are likely to exert their therapeutic effect by inhibiting the production or activity of IL-1α or IL-1β—it is also possible that they exert an associated or additional beneficial effect by stimulating the production of anti-inflammatory cytokine(s), such as interleukin-10 (IL-10).

According to the present invention there is provided a polypeptide capable of ameliorating an inflammatory skin condition wherein said polypeptide is a modified thioredoxin, the modification comprising:
a. substituting Cys₁ and Cys₂ in the motif Cys₁-Gly-Pro-Cys₂ present in the unmodified thioredoxin with an amino acid other than cysteine with the proviso that if one Cys is substituted with Ser the other Cys is not substituted with Ser; or
b. substituting either of Cys₁ and Cys₂ in the motif Cys₁-Gly-Pro-Cys₂ present in the unmodified thioredoxin with an amino acid other than cysteine and deleting the non-substituted cysteine.

Preferably, the modification consists of independently substituting both Cys₁ and Cys₂ with an amino acid other than Ser. The modification of the active site renders the active site redox-inactive and, surprisingly, it has been found that such redox-inactive molecules are capable of ameliorating an inflammatory skin condition.

The present invention further provides a modified thioredoxin wherein if the unmodified thioredoxin contains one or more cysteines in addition to Cys₁ and Cys₂, then the modification further comprises substituting and/or deleting one or more of the additional cysteines.

Both Cys₁ and Cys₂ may be independently substituted. For example, one embodiment of the polypeptide of the present invention could comprise Ser-Gly-Pro-Ala, another Ala-Gly-Pro-Ser. In a preferred embodiment of the invention Cys₁ and Cys₂ are both substituted by Ala to give Ala-Gly-Pro-Ala. More preferred is a polypeptide wherein the unmodified TRX is human TRX, and more preferred still is the polypeptide selected from the group consisting of SEQ ID NO. 3, SEQ ID NO. 9 and SEQ ID NO. 10.

A further embodiment of the present invention is a DNA sequence that encodes a polypeptide of the present invention. The exact nature of the DNA sequence would, of course, depend on the specific nature of the polypeptide and the intended use of the DNA sequence. For example, codon-optimisation of the DNA sequence may be required for expression of the DNA sequence in a recombinant expression system (an example of a codon-optimised sequence is provided as SEQ ID NO. 6). The techniques required to provide such DNA sequences are well within the knowledge of the skilled man. A preferred DNA sequence of the present invention is depicted in SEQ ID NO. 4.

The present invention also relates to the use of the polypeptides of the present invention as a pharmaceutical—and a pharmaceutical composition/medicament—suitable for treating inflammatory skin conditions preferably comprising the polypeptide(s) of the present invention. For therapeutic purposes the polypeptide(s) of the present invention may be administered by any conventional means, either as an individual therapeutic agent or in combination with other therapeutic agents. The pharmaceutical compositions of the present invention can be adapted, using methods well known to those skilled in the pharmaceutical art, depending on the exact route of administration desired. Compositions of the present invention include, but are not limited to, those suitable for application to the skin via, for example, topical application and subcutaneous application. For the treatment of psoriasis, topical application is sufficient to give a therapeutic effect.

The present invention further relates to methods of producing the polypeptide of the present invention. Such methods would include recombinant expression of said polypeptide and in particular transforming an organism with a vector comprising a DNA sequence encoding the polypeptide, wherein said vector is capable of expressing said DNA sequence in said organism and growing said organism in conditions which allow the expression of said DNA sequence to produce said polypeptide. By growing it is meant increasing biomass, for example where the organism is a unicellular organism growing means increasing cell number. The term “organism” includes any organism that is suitable for the recombinant expression of the polypeptides of the present invention. Suitable recombinant expression systems include, but are not limited to, mammalian cell cultures, yeast and bacteria. Particularly preferred is E.coli. Vectors suitable for expression in host cell such as these would be readily apparent to the skilled man and include, for example vectors that harbour the T7 promoter, such as pET vectors, for expression in E. coli and other vectors suitable for expression in the yeast Pichia pastoris. The method of producing the polypeptide may also include the purification of the polypeptide. By purification it is meant obtaining the recombinant polypeptide from the production materials. Methods such as these could be employed during the Good Manufacturing Practice (GMP) production of these polypeptides.

The present invention further relates to a method of ameliorating an inflammatory skin condition comprising applying to a skin surface an effective amount of a composition comprising a molecule selected from the group consisting of:
a. a protein comprising a thioredoxin active site (Cys₁-Gly-Pro-Cys₂);
b. a thioredoxin (TRX);
c. a modified thioredoxin wherein said modification comprising substituting and/or deleting at least one of the cysteines present in the unmodified thioredoxin with an amino acid other than cysteine;
d. a polypeptide according to the present invention; and
e. a molecule that comprises a region of three dimensional similarity to a region present within the three dimensional structure of the protein depicted in SEQ ID NO. 1, and which is capable of ameliorating an inflammatory skin condition.

The term “inflammatory skin condition” includes, for example, a human inflammatory skin condition and an animal inflammatory skin condition. In a preferred embodiment the inflammatory skin condition is selected from the group consisting of psoriasis, lichen planus, atopic eczema, irritant or allergic contact dermatitis, contact urticaria, infantile eczema and acne. The methods of the present invention are also useful in assisting wound healing, and in the treatment of burns, especially sunburn. Psoriasis is a chronic inflammatory skin condition characterised by the appearance of discrete psoriatic plaques. Psoriasis is associated with a number of changes in skin morphology. There is increasing evidence that pro-inflammatory cytokines play important roles in the pathogenesis of psoriasis. Current treatments include local topical administration of anti-inflammatory agents—typically a corticosteroid. Such treatments are not fully effective and are associated with unwanted side effects. Another therapeutic strategy is disruption of TNF-α function, but this also has been found to cause adverse reactions. There is a need therefore to provide further molecules that are effective in the treatment of inflammatory skin disorders, but which exhibit little or no adverse side effects and which ideally can be delivered by a non-invasive method, for example by application directly onto the inflammation.

Preferred for use in the method of the present invention is a molecule capable of inhibiting production and/or activity of IL-1α and/or IL-1β and/or capable of stimulating or enhancing the production and/or activity of IL-10.

TRX is a small (10-14 kDa), ubiquitous protein that is an important component of the cellular redox regulation system. Suitable TRX for use in the method of the present invention include TRX from (1) a prokaryote (e.g E. coli—SEQ ID NO. 7), (2) a plant (e.g Arabidopsis—SEQ ID NO. 8) and (3) an animal (e.g human—SEQ ID NO. 1). TRX can exist in a reduced state (wherein the two cysteines at the active site (Cys₁-Gly-Pro-Cys₂) provide a dithiol) and an oxidised state (wherein there is a disulphide bridge formed between the two cysteines at the active site). Under physiological conditions both redox states can exist—and both forms can be utilised in respect of the present invention. Furthermore, it is known that certain thioredoxins can exist in multimeric forms. For example, it is known that human TRX (hTRX) can form dimers wherein a disulphide bridge exists between Cys-73. These multimeric forms of the molecules may also be utilised, in addition to the monomeric form, within the methods of the present invention. It is however, preferred, that the molecule, for example thioredoxin, is in a substantially reduced state. By substantially reduced it is meant that >80%, preferably >90%, more preferably >95% of the molecules present are in a reduced state. A preferred molecule for use in the method is the recombinant human thioredoxin depicted in SEQ ID NO. 1—since this protein is an endogenous human protein, and is therefore unlikely to cause either adverse effects, or an immune response when administered to patients. Other molecules suitable for use in the method of the present invention include a protein that comprises a thioredoxin active site in which one or both of the cysteines at the active site are replaced by an amino acid other than cysteine. Examples wherein one or other of the cysteines is replaced include Cys₁-Gly-Pro-Ala and Ala-Gly-Pro-Cys₂. Surprisingly, it has been discovered that redox-inactive TRX molecules, in which both Cys₁ and Cys₂ are replaced, can also be successfully used in the present inventive methods. Furthermore, it has been shown that where the TRX molecule comprises additional cysteines other than at the active site these additional cysteines can also be replaced without any loss in activity. For example, in respect of human thioredoxin, which contains five cysteines (C₃₂, C₃₅, C₆₂, C₆₉ and C₇₃) it has been shown that modified human thioredoxins comprising (1) C73A, (2) C32A, C35A and C73A; and (3) C32A, C35A, C62A, C69A and C73A retain biological activity. It has also been shown that activity is retained if cysteines present in the unmodified thioredoxin other than the active site cysteines are substituted and/or deleted. For example, the protein depicted in SEQ ID NO. 11 (C73A) has been shown to be active. It has also been found that the active molecules can be rendered inactive by heat treatment at 95° C. for 30 min, or 56° C. for 30 min, indicating that there is a structural feature associated with these molecules that is responsible for the observed activity. Thus the present invention further relates a molecule which comprises a region of three dimensional homology to a region present within the three dimensional structure of the active molecules disclosed in the present application, for example SEQ ID NO. 1, that are capable of ameliorating an inflammatory skin condition. Particularly preferred for use in the method are the polypeptides of the present invention, including the polypeptide sequences depicted in SEQ ID NO. 3, SEQ ID NO. 9 SEQ ID NO. 10 and SEQ ID NO. 11.

It has been shown that the molecule(s) described can be used to treat inflammatory skin conditions at extremely low application rates. Accordingly, the present invention further provides a pharmaceutical composition wherein the concentration of the active molecule within the pharmaceutical composition is preferably from 0.0001 to 0.5 w/v (1 μg/ml to 5 mg/ml) more preferably 0.0001 to 0.1% w/v, more preferably 0.0001% to 0.01% w/v, and still more preferably 0.0001% to 0.001% w/v. If the composition is a cream then it is particularly preferred that the active molecule is present at a concentration from 0.0001% to 0.02% w/v. Compositions comprising recombinant human thioredoxin in a substantially reduced, monomeric state are particularly preferred.

The application rate of the molecules described above to the skin surface is preferably 0.05 to 10 μg /cm², more preferably 0.05 to 5 μg/cm², and more preferably 0.1 to 1 μg/cm². It is preferred that human thioredoxin in a substantially reduced, monomeric state is applied to the skin surface.

The present invention further relates to a method of treating inflammatory skin conditions comprising applying to a skin surface an effective amount of a composition comprising a molecule described above and an additional active ingredient. By additional active it is meant an ingredient that also has a pharmaceutical effect—which could be either additive or synergistic to the said molecule. Examples of additional active ingredients include lactoferrin (e.g. as depicted in SEQ ID NO. 5) and/or corticosteroids. The present invention also relates to a pharmaceutical composition comprising a molecule described above and an additional active ingredient. Preferred additional active ingredients include lactoferrin (e.g. as depicted in SEQ ID NO. 5), and/or corticosteroids and/or other topical medicaments suitable for the treatment of inflammatory skin conditions. A preferred composition is wherein the molecule is human thioredoxin depicted in SEQ ID NO. 1 and/or the modified TRX depicted in SEQ ID NO. 3 and wherein the additional active ingredient is lactoferrin, depicted in SEQ ID NO. 5. The compositions of the present invention may also comprise further ingredients, for example anti-oxidants such as glutathione, vitamin A, vitamin C, vitamin E, or indeed extracts from plants such as, for example, Aloe vera. The pharmaceutical compositions of the present invention can also be used in a combination therapy for the treatment of severe inflammatory skin conditions.

It is preferred that composition of the present invention is suitable for application to the skin. Accordingly the composition will typically be formulated as a solution, gel, lotion, ointment, cream, suspension, paste, liniment, powder, tincture, aerosol, transdermal drug delivery system, or similar in a pharmaceutically acceptable form by methods well known in the art. Substances that enhance the penetration of the active ingredients through the skin may also be added including, for example, dimethylsulfoxide, dimethylacetamide, dimethylformamide, surfactants, azone, alcohol, acetone, propylene glycol and polyethylene glycol. The compositions may be applied directly to the skin or via various transdermal drug delivery systems, such as patches.

The present invention further relates to the use of a polypeptide capable of ameliorating an inflammatory skin condition wherein said polypeptide is a modified thioredoxin, the modification comprising:
a. substituting Cys₁ and Cys₂ in the motif Cys₁-Gly-Pro-Cys₂ present in the unmodified thioredoxin with an amino acid other than cysteine; or
b. substituting either of Cys₁ and Cys₂ in the motif Cys₁-Gly-Pro-Cys₂ present in the unmodified thioredoxin with an amino acid other than cysteine and deleting the non-substituted cysteine;
   as a pharmaceutical.

The present invention further relates to the use of the polypeptide depicted in SEQ ID NO.s 11 and 17 as a pharmaceutical.

The present invention further relates to the use of a polypeptide capable of ameliorating an inflammatory skin condition wherein said polypeptide is a modified thioredoxin, the modification comprising:
a. substituting Cys₁ and Cys₂ in the motif Cys₁-Gly-Pro-Cys₂ present in the unmodified thioredoxin with an amino acid other than cysteine; or
b. substituting either of Cys₁ and Cys₂ in the motif Cys₁-Gly-Pro-Cys₂ present in the unmodified thioredoxin with an amino acid other than cysteine and deleting the non-substituted cysteine.
   in the manufacture of a medicament suitable for application to a skin surface for ameliorating an inflammatory skin condition; the use of thioredoxin in the manufacture of a medicament suitable for application to a skin surface for ameliorating an inflammatory skin condition; and the use of human thioredoxin depicted in SEQ ID NO. 1 in the manufacture suitable for application to a skin surface for ameliorating an inflammatory skin condition.

The present invention further relates to the use of the polypeptide depicted in SEQ ID NO.s 11 and 17 in the manufacture of a medicament suitable for application to a skin surface for ameliorating an inflammatory skin condition.

### List of Sequences

All sequences are provided herewith with an N-terminal methionine. For the avoidance of doubt, it should be understood that the present invention also includes sequences wherein the N-terminal methionine is absent.
SEQ ID NO. 1 Human TRX (protein)
SEQ ID NO. 2 Human TRX (DNA)
SEQ ID NO. 3 Modified Human TRX (protein)
SEQ ID NO. 4 Modified Human TRX (DNA)
SEQ ID NO. 5 Human Lactoferrin (protein)
SEQ ID NO. 6 DNA sequence encoding human TRX optimised. for expression in E. coli.
SEQ ID NO. 7 E.coli thioredoxin.
SEQ ID NO. 8 Arabidopsis thioredoxin
SEQ ID NO. 9 Triple modified human thioredoxin (C32A, C35A, C73A).
SEQ ID NO. 10 Cysteine free human thioredoxin (C32A, C35A, C62A, C69A, C73A).
SEQ ID NO. 11 Modified Human TRX (C73A).
SEQ ID NO. 12 Modified Human TRX (C32S).
SEQ ID NO. 13 Modified Human TRX (C35S).
SEQ ID NO. 14 Modified Human TRX (C32S C35S).
SEQ ID NO. 15 Modified Human TRX (C32S C69S).
SEQ ID NO. 16 Modified Human TRX (C35S C69S).
SEQ ID NO. 17 Modified Human TRX (C73S)

### LIST OF FIGURES

The terms “TRX” and “Thio” are both used interchangeably as abbreviations for thioredoxin.

FIG. 1. Groups of mice (n=3) received 30 μl of aqueous cream (cr) or 30 μl of native human TRX (0.5 μg; TRX—SEQ ID NO. 1) on the dorsum of both ears. Two hours later, mice were exposed topically on the dorsum of both ears to 0.5% oxazolone (Ox) or to vehicle alone (acetone:olive oil; AOO). Control mice were untreated (naïve; −). Epidermal sheets were prepared for analysis of major histocompatibility complex (MHC) class II, (Ia)⁺ LC frequencies 4 h later. LC numbers (mean±SE) are derived from analysis of n=6 epidermal sheets/treatment group.

FIG. 2. Groups of mice (n=3) received 30 μl of aqueous cream (cr) or 30 μl of native human TRX (0.5 μg; Thio) on the dorsum of both ears. Two hours later, mice received 50 ng of murine TNFα or IL-1β by intradermal injection into ear pinnae. Control mice were untreated (naïve; −). Epidermal sheets were prepared for analysis of MHC class II (Ia)⁺ LC frequencies 4 h (IL-1b) or 30 min (TNFα) later. LC numbers (mean±SE) are derived from analysis of n=6 epidermal sheets/treatment group.

FIG. 3. Groups of mice (n=10) received 30 μl of aqueous cream (cr) or 30 μl of native human TRX (0.5 μg; Thio) on the dorsum of both ears. Two hours later, mice received 50 ng of murine TNFα or IL-1β by intradermal injection into ear pinnae. Control mice were untreated (naïve). Draining auricular lymph nodes were excised 17 h (IL-1β) or 4 h (TNFα) later, pooled for each experimental group and a single cell suspension of LNC prepared. DC were enriched by density gradient centrifugation. DC numbers were assessed following direct morphological examination of DC-enriched fractions and are expressed as number of DC per node.

FIG. 4. Groups of mice (n=3) received 30 μl of aqueous cream (cr), 30 μl of native human TRX (0.5 μg; hTRX) or 30 μl of modified human TRX (0.5 μg; C32AC35A—SEQ ID NO. 3) on the dorsum of both ears. Two hours later, mice were exposed topically on the dorsum of both ears to 0.5% oxazolone (Ox). Control mice were untreated (naïve). Epidermal sheets were prepared for analysis of MHC class II (Ia)⁺ LC frequencies 4 h later. LC numbers (mean±SE) are derived from analysis of n=6 epidermal sheets/treatment group.

FIG. 5. Groups of mice (n=3) received 30 μl of aqueous cream (cr), 30 μl of native human TRX (0.5 μg; hTRX) or 30 ml of various amounts of modified human TRX (0.5, 0.1 or 0.05 μg; C32AC35A—SEQ ID NO. 3) on the dorsum of both ears. Two hours later, mice were exposed topically on the dorsum of both ears to 0.5% oxazolone (Ox). Control mice were untreated (naïve). Epidermal sheets were prepared for analysis of MHC class II (Ia)⁺ LC frequencies 4 h later. LC numbers (mean±SE) are derived from analysis of n=6 epidermal sheets/treatment group.

FIG. 6. Groups of mice (n=3) received 30 μl of aqueous cream (cr), 30 μl of modified human TRX (0.5 μg; C32AC35A—SEQ ID NO.3) or 30 μl of various amounts native human TRX (0.5, 0.1 or 0.05 μg; hTRX) on the dorsum of both ears. Two hours later, mice were exposed topically on the dorsum of both ears to 0.5% oxazolone (Ox). Control mice were untreated (naive). Epidermal sheets were prepared for analysis of MHC class II (Ia)⁺ LC frequencies 4 h later. LC numbers (mean±SE) are derived from analysis of n=6 epidermal sheets/treatment group.

FIG. 7. Healthy volunteers (a and b) were exposed topically at two sites to native human TRX (Trx; 0.5μg in 50 μl) and at a further two sites to an equivalent volume of aqueous cream alone. Two hours later, human recombinant TNF-α (500 U) or an equal volume of saline was injected intradermally into paired sites (one pre-treated with Trx and one with cream) and biopsies taken 2 h later. CD1a⁺ LC densities were assessed following indirect immunofluorescence staining of epidermal sheets. Results are expressed as the mean±SD number of cells/mm² derived from examination of 50 fields/sample.

FIG. 8. Graph indicating that the modified human TRX (C32A/C35A—SEQ ID NO. 3) is redox-inactive.

FIG. 9. Groups of mice (n=5) received 30 μl of aqueous cream (cr), 30 μl of native human thioredoxin (0.5 μg; hTRX) of on the dorsum of both ears. Two hours later, mice were exposed topically on the dorsum of both ears to 0.5% oxazolone (Ox). Control mice received an equal volume of vehicle (AOO) alone. Two hours later, ears were excised and explants prepared and cultured for 16 h at 37° C. IL-10 content was analyzed by Bioplex cytokine array and results are expressed as pg/ml IL- 10 produced per mouse.

FIG. 10. Inhibition of oxazolone-induced LC migration by the monomeric hTRX mutant C73A in mice. Groups of mice (n=3) were exposed topically on the dorsum of both ears to 30 μl aqueous cream BP containing 0.5 μg oligomeric hTRX, 0.5 μg C73A or cream alone, 2 h prior to application at the same site of 0.5% oxazolone (Ox) suspended in vehicle (4:1 acetone:olive oil). Control mice were untreated (naïve). After 4 h, ears were removed and epidermal sheets were prepared from dorsal ear halves for indirect immunofluorescence staining for MHC class II (Ia) expression. Results are displayed as the mean number (±SE) of Ia⁺ LC/mm² of epidermis following examination of 10 fields/ear for each of 6 ears.

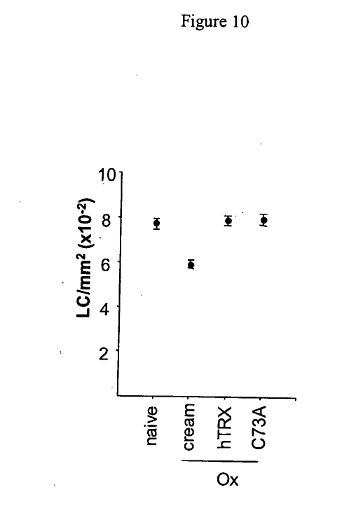

FIG. 11. Inhibition of oxazolone-induced LC migration by the cysteine-free hTRX mutant (C32AC35AC62AC69AC73A) in mice. Groups of mice (n=3) were exposed topically on the dorsum of both ears to 30 μl aqueous cream BP containing 0.5 μg oligomeric hTRX, 0.5 μg C32AC35AC62AC69AC73A (Cys-free) or cream alone, 2 h prior to application at the same site of 0.5% oxazolone (Ox) suspended in vehicle (4:1 acetone:olive oil). Control mice were untreated (naïve). After 4 h, ears were removed and epidermal sheets were prepared from dorsal ear halves for indirect immunofluorescence staining for MHC class II (Ia) expression. Results are displayed as the mean number (±SE) of Ia⁺ LC/mm of epidermis following examination of 10 fields/ear for each of 6 ears.

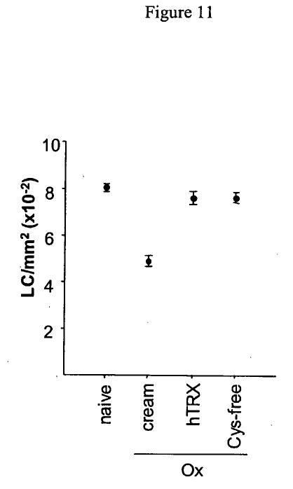

FIG. 12. Inhibition of oxazolone-induced LC migration by the triple mutant C32AC35AC73A in mice. Groups of mice (n=3) were exposed topically on the dorsum of both ears to 30 μl aqueous cream BP containing 0.5 μg oligomeric hTRX, 0.5 μg C32AC35AC73A or cream alone, 2 h prior to application at the same site of 0.5% oxazolone (Ox) suspended in vehicle (4:1 acetone:olive oil). Control mice were untreated (naïve). After 4 h, ears were removed and epidermal sheets were prepared from dorsal ear halves for indirect immunofluorescence staining for MHC class II (Ia) expression. Results are displayed as the mean number (±SE) of Ia⁺ LC/mm² of epidermis following examination of 10 fields/ear for each of 6 ears.

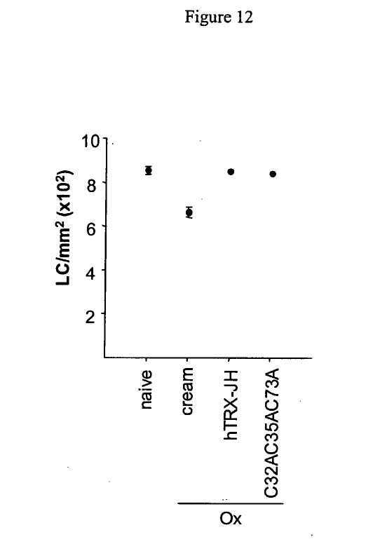

FIG. 13. Influence of heat treatment (95° C. for 30 min) on inhibition of oxazolone-induced LC migration by oligomeric hTRX in mice. Groups of mice (n=3) were exposed topically on the dorsum of both ears to 30 μl aqueous cream BP containing 0.5 μg oligomeric hTRX, 0.5 μg heat treated (95° C. for 30 min) oligomeric hTRX (hTRX-HT) or cream alone, 2 h prior to application at the same site of 0.5% oxazolone (Ox) suspended in vehicle (4:1 acetone:olive oil). Control mice were untreated (naïve). After 4 h, ears were removed and epidermal sheets were prepared from dorsal ear halves for indirect immunofluorescence staining for MHC class II (Ia) expression. Results are displayed as the mean number (±SE) of Ia⁺ LC/mm² of epidermis following examination of 10 fields/ear for each of 6 ears.

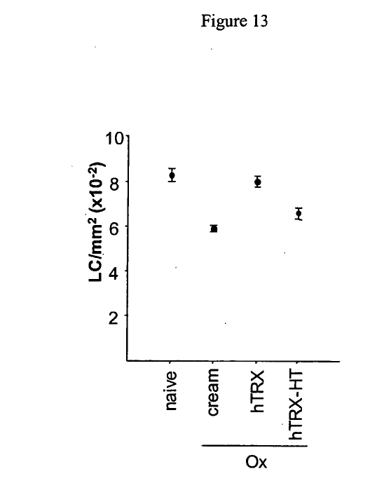

FIG. 14. Influence of heat treatment (56° C. for 30 min) on inhibition of oxazolone-induced LC migration by oligomeric hTRX in mice. Groups of mice (n=3) were exposed topically on the dorsum of both ears to 30 μl aqueous cream BP containing 0.5 μg oligomeric hTRX, 0.5 μg heat treated (56° C. for 30 min) oligomeric hTRX (hTRX-HT) or cream alone, 2 h prior to application at the same site of 0.5% oxazolone (Ox) suspended in vehicle (4:1 acetone:olive oil). Control mice were untreated (naïve). After 4 h, ears were removed and epidermal sheets were prepared from dorsal ear halves for indirect immunofluorescence staining for MHC class II (Ia) expression. Results are displayed as the mean number (±SE) of Ia⁺ LC/mm² of epidermis following examination of 10 fields/ear for each of 6 ears.

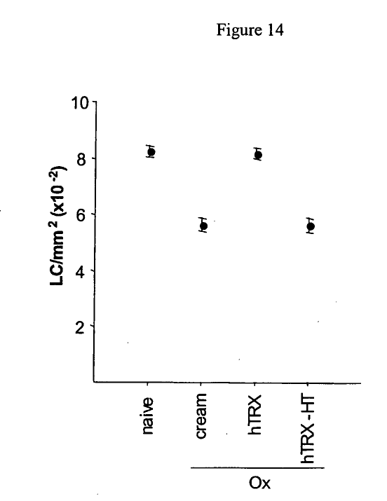

FIG. 15. Inhibition of oxazolone-induced LC migration in mice by reduced monomeric hTRX. Groups of mice (n=3) were exposed topically on the dorsum of both ears to 30 μl aqueous cream BP containing various concentrations of hTRXrm (0.5 μg, 4 μg, 20 μg), or cream alone, 2 h prior to application at the same site of 0.5% oxazolone (Ox) suspended in vehicle (4:1 acetone:olive oil). Control mice were untreated (naïve). After 4 h, ears were removed and epidermal sheets were prepared from dorsal ear halves for indirect immunofluorescence staining for MHC class II (Ia) expression. Results are displayed as the mean number (±SE) of Ia⁺ LC/mm² of epidermis following examination of 10 fields/ear for each of 6 ears.

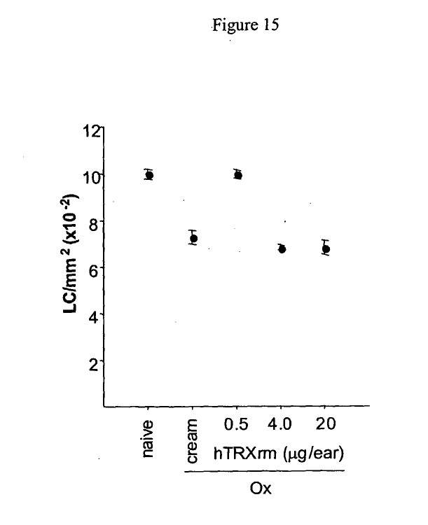

### EXPERIMENTS

Mouse studies

Mice

Young adult (6- to 8-week old) male BALB/c strain mice obtained from the Specific Pathogen Free Breeding Unit (Alderley Park, Cheshire, UK) were used throughout these investigations.

### Thioredoxin

Recombinant native human TRX (hTRX—SEQ ID NO. 1) or modified human TRX (SEQ ID NO. 3) were diluted to 16.7 μg/ml in aqueous cream BP and 30 μl (0.5 μg TRX) applied topically to the dorsum of both ears 2 hours prior to exposure at the same site to chemical or cytokine. Control mice received an equivalent volume of cream alone. In some experiments, animals received 0.5, 0.1 and 0.05 μg of TRX.

Chemicals and Exposure

The skin sensitising chemical 4-ethoxy-2-phenyloxazol-5-one (oxazolone; Sigma Chemical Co., St Louis, Mo.) was dissolved in 4:1 acetone:olive oil (AOO). Groups of mice received 25 μl of 0.5% oxazolone, or vehicle (AOO) alone, on the dorsum of both ears. Other control animals were untreated (naïve).

### Cytokines

Recombinant murine TNF-α (specific activity 2×10⁸ U/mg by L929 cytotoxicity assay; endotoxin level: 0.009 ng/μg) was obtained from Genzyme (West Malling, Kent, UK). Recombinant murine IL-10 (specific activity 1-2×10⁸ U/mg; endotoxin level: <0.1 ng/μg) was purchased from R&D Systems (Oxon, UK). Cytokines were either supplied as, or reconstituted in, sterile solutions of phosphate buffered saline (PBS) containing 0.1% bovine serum albumin (BSA) as carrier protein. Cytokines were diluted with sterile PBS containing 0.1% BSA and were administered using 1 ml syringes with 30-gauge stainless steel needles. Mice received 30 μl intradermal injections into both ear pinnae.

Preparation and Analysis of Epidermal Sheets

Ears were removed either 4 h following exposure to chemical or IL-1β, or 30 min after treatment with TNF-α. Samples were split with the aid of forceps into dorsal and ventral ear halves. The dorsal halves were incubated for 90 min at 37° C. with 0.02M ethylenediamine tetra-acetic acid (EDTA; Sigma) dissolved in PBS. The epidermis was separated from the dermis using forceps and washed in PBS. Epidermal sheets were fixed in acetone for 20 min at −20° C. Following fixation, sheets were washed in PBS and then incubated at room temperature for 30 min with anti-mouse MHC (I-A^{d}/I-E^{d}) monoclonal antibody diluted to 5 μg/ml in 0.1% BSA/PBS. Sheets were then washed prior to incubation for a further 30 min with FITC-conjugated F(ab)₂ goat anti-rat IgG, diluted 1:100 in 0.1% BSA/PBS. Finally, sheets were washed in PBS and mounted on microscope slides in Citifluor (Citifluor Ltd., London, UK) and sealed with nail varnish. Samples were examined in a blinded fashion by fluorescence microscopy and the frequency of stained cells assessed using an eyepiece with a calibrated grid (0.32×0.213 at ×40 magnification). For each sample 10 consecutive fields in the central portion of the ear were examined.

Measurement of Epidermal Cytokine Production

Ears were removed 2 h following exposure to 0.5% oxazolone and prepared for explant culture under aseptic conditions. Ears were washed immediately in 70% ethanol, rinsed in PBS and were split with the aid of forceps into dorsal and ventral halves. Dorsal halves were floated on 250 μl RPMI-1640 medium in 24-well tissue culture plates (1 dorsal ear half/well). Supernatants were collected after 16 h of culture, pooled for each mouse and centrifuged at 150 g for 5 min prior to storage at −70° C. The IL-10 content was measured in supernatants using the Bio-Plex™ cytokine array system according to manufacturer's instructions (Bio-Rad Laboratories, Hercules, Calif., USA).

Preparation and Analysis of Dendritic Cells (DC)

Draining auricular lymph nodes were excised 18 h following treatment with chemical, or 4 h and 17 h following administration of the cytokines TNF-α and IL-1β, respectively. Nodes were pooled for each experimental group. A single cell suspension of lymph node cells (LNC) was prepared under aseptic conditions by mechanical disaggregation through sterile 200-mesh stainless steel gauze and resuspended in RPMI-1640 growth medium (Gibco, Renfrewshire, UK) supplemented with 25 mM HEPES, 400 μg/ml streptomycin, 400 μg/ml ampicillin and 10% heat-inactivated fetal calf serum (RPMI-FCS). Viable cell counts were performed by exclusion of trypan blue dye and the total cellularity per lymph node recorded. The cell concentration was adjusted to 5×10⁶ cells/ml in RPMI-FCS and DC-enriched populations were prepared by discontinuous gradient centrifugation on Metrizamide (Sigma Chemical Co.; 14.5% in RPMI-FCS). The frequency of DC in such low buoyant density fractions was assessed routinely by direct morphological examination using phase contrast microscopy.

### Human Studies

### TRX and Exposure

TRX in aqueous cream (0.5 μg in 50 μl) was applied topically to two skin sites (each 2 cm² area) identified on non-sun-exposed buttock or hip. A further two sites on the contralateral buttock or hip received 50 μl of aqueous cream alone. Two hours later, volunteers received two 50 μl intradermal injections of 500 U of homologous recombinant TNF-α diluted in sterile normal saline and two control injections of 50 μl of sterile saline alone to paired sites (one exposed previously to TRX and one exposed to cream alone). Punch biopsies (6 mm) were taken under local anaesthesia (1% lignocaine) from each of the treated sites 2 h later.

### Preparation and Analysis of Epidermal Sheets

Epidermal Langerhans cells (LC) were identified on the basis of their expression of CD1a; a membrane determinant that characterises LC in human epidermis. To stain for LC, biopsies were placed immediately in 0.02M ethylenediamine tetraacetic acid (Sigma, St Louis, Mo., USA) dissolved in phosphate buffered saline (PBS) and incubated for 2 heat 37° C. The epidermis was separated from the dermis using forceps, washed in PBS and fixed in acetone at −20° C. After washing in PBS, epidermal sheets were incubated at room temperature for 30 min with monoclonal antibodies specific for CD1a [clone NA1/34 (mouse IgG2a); DAKO Ltd, Cambridge, UK] diluted to 10 μg/ml in PBS containing 0.1% bovine serum albumin (BSA). Sheets were washed prior to incubation for a further 30 min with fluorescein isothiocyanate-conjugated goat F(ab′)₂ anti-mouse immunoglobulins (DAKO) diluted 1:100 in 0.1% BSA/PBS. Finally, sheets were washed in PBS and mounted on microscope slides in Citifluor (Citifluor Ltd., London, UK) and sealed with nail varnish. The identity of each slide was then masked using tape.

Samples were examined by fluorescence microscopy and the frequency of stained cells assessed in a blinded fashion using an eyepiece with a calibrated grid (0.32×0.213 mm at ×40 magnification). For each sample, 50 consecutive fields were examined. The identity of each slide was revealed after all samples have been counted. Results are expressed as the mean±SD number of cells/mm².

### Experiment 1

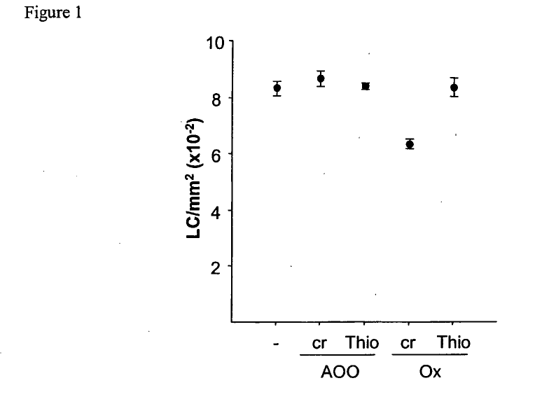

The purpose this experiment was to determine whether topical application of native hTRX to mouse skin was able to influence the integrity of LC migration induced by subsequent exposure at the same site to oxazolone, a potent contact allergen. The results of a representative experiment are illustrated in FIG. 1. The results reveal that prior exposure to hTRX causes a complete inhibition of allergen-induced LC migration. The conclusion drawn is that topically applied hTRX is able to reach the viable epidermis of mouse skin at concentrations sufficient to inhibit one or more biological processes required for the effective mobilisation and migration of LC in response to a stimulus, in this instance a contact allergen.

### Experiment 2

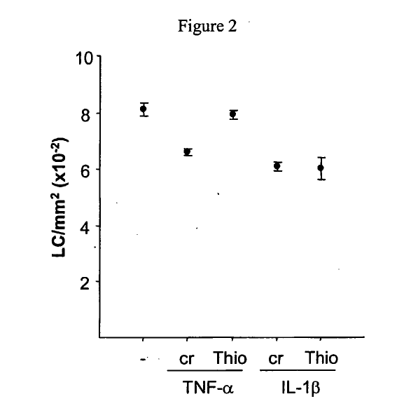

Previous studies have provided clear evidence that the migration of epidermal LC, in both mouse and man, is dependent upon the availability of certain cytokines and chemokines, two of those known to be of particular importance being interleukin- 1β (IL-1β) and tumour necrosis factor α (TNF-α). There is a precedent for perturbation of cytokine function resulting in compromised LC migration. In the next experiments we therefore investigated whether hTRX could affect LC migration induced by either IL-1β or TNF-α. The results of a representative experiment are displayed in FIG. 2. These data reveal that prior topical exposure of mice to hTRX was able to cause an almost complete inhibition of LC migration induced by the intradermal (id) injection of homologous TNF-α. In contrast, hTRX applied in the same way was without influence on the integrity of LC migration provoked by id administration of homologous IL-1β. The interpretation is that topical administration of hTRX is associated with a perturbation of IL-1β function. Thus, hTRX was able to inhibit very effectively LC mobilisation in response to either allergen (oxazolone) (FIG. 1), or TNF-α (FIG. 2) in both of which circumstances there is a requirement for the availability of bioactive IL-1β. However, the inhibitory effects of hTRX can be overcome by the addition of an exogenous source of IL-1β in which case the effectiveness of migration is unimpaired.

### Experiment 3

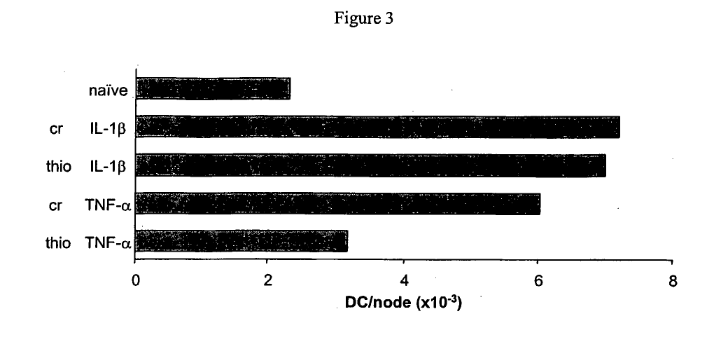

In a parallel series of experiments the same question as addressed in Experiment 2 was explored, but using a supplementary endpoint. In this case the endpoint used was the accumulation of dendritic cells (DC) in skin-draining regional lymph nodes. The relevance of this measurement is that the epidermal LC that are provoked to migrate from the skin traffic via afferent lymphatics to draining lymph nodes (in order to interact with the adaptive immune system). The effectiveness of LC mobilisation can therefore be measured either as a function of the loss of LC from the epidermis, or as a function of their subsequent accumulation in skin-draining lymph nodes. A representative experiment is illustrated in FIG. 3 where the impact of hTRX on DC accumulation in lymph nodes following id administration of either IL-1β or TNF-α has been examined. The results are consistent with those shown in FIG. 2. That is, hTRX was found to inhibit DC accumulation in response to TNF-α, but not in response to IL-1β

### Experiment 4

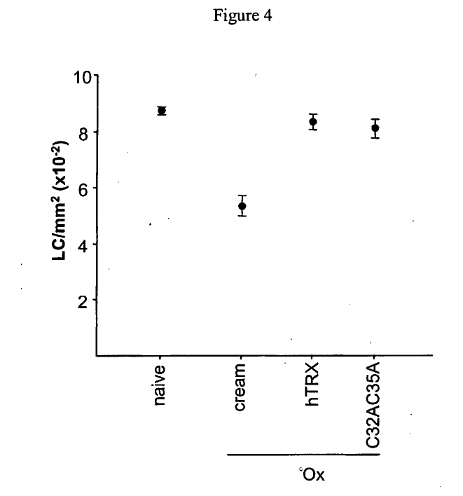

Most biological properties of TRX are considered to be a function of the redox activity of this protein. There are available redox-inactive mutant variants of the protein that have discrete amino substitutions that render the protein redox-inactive. One such mutant is C32A/C35A, as depicted in SEQ ID NO. 3. In another series of experiments the ability of C32A/C35A to inhibit LC migration was investigated and compared with the activity of native hTRX (SEQ ID NO. 1). A representative experiment is shown in FIG. 4. In these experiments LC mobilisation was stimulated with the chemical allergen oxazolone and the ability of either hTRX or C32A/C35A to inhibit this response was measured. The results summarised in FIG. 4 demonstrate clearly that both native hTRX and the redox-inactive mutant C32A/C35A are able to inhibit very substantially the integrity of LC migration. The conclusion drawn is that the effects of TRX on LC migration (and the integrity of IL-1β signalling) are independent of active redox function.

### Experiment 5

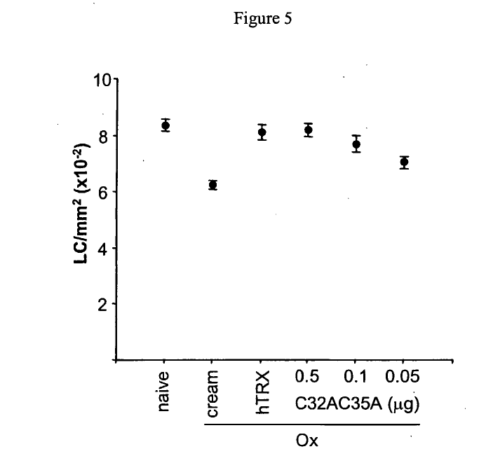

In subsequent experiments the relative potency of native hTRX (SEQ ID NO. 1) and of C32A/C35A (SEQ ID NO. 3) were compared with respect to inhibition of LC migration. In one experimental design various concentrations of the redox-inactive mutant protein were compared with a single concentration of the native hTRX. The results of a representative experiment are summarised in FIG. 5. The data available reveal a dose-dependent inhibition of LC migration. Exposure of mice to 0.5 μg of C32A/C35A (or to 0.5 μg of native hTRX) was characterised by a complete inhibition of allergen-induced LC migration. Although lower concentrations of C32A/C35A (0.1 μg or 0.05 μg) were able to inhibit allergen-induced LC migration their effects were less complete than that seen with the higher dose of protein.

### Experiment 6

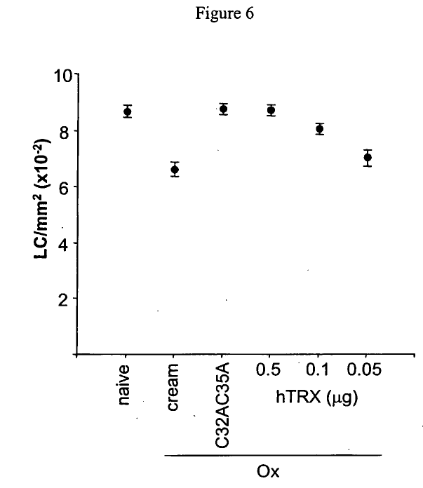

In parallel investigations the same experimental design was employed with the reverse orientation. That is, a dose response was performed with the native hTRX and the results compared with the effects of a single dose of the redox-inactive mutant. A representative experiment is illustrated in FIG. 6. Again, a clear dose response relationship was observed. Treatment of mice with 0.5 μg of hTRX (or with 0.5 μg of C32A/C35A) caused a complete inhibition of allergen-induced LC migration. Lower doses of hTRX (0.1 μg or 0.05 μg) although having some effect, caused a less complete inhibition of migration than did 0.5 μg. Taken together these data confirm that hTRX and C32A/C35A both cause an inhibition of LC migration, and do so with comparable potency.

### Experiment 7

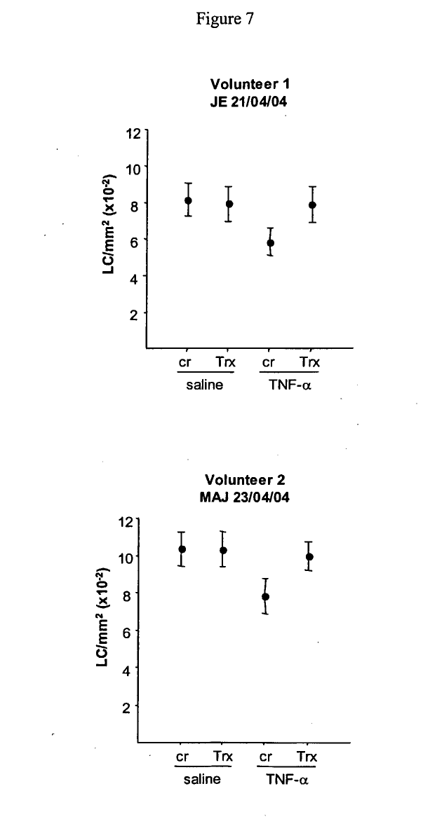

In the next series of experiments the impact of hTRX on the integrity of LC migration in humans was investigated using healthy adult volunteers. The results obtained using two such volunteers are illustrated in FIG. 7. In common with previous studies conducted in mice (see FIG. 2 above), it was observed in each of the two volunteers that prior topical exposure to hTRX caused a significant inhibition of LC migration stimulated subsequently by the id administration of homologous recombinant TNF-α. These data confirm that hTRX effects changes in human skin comparable to those observed initially in mouse skin.

### Experiment 8

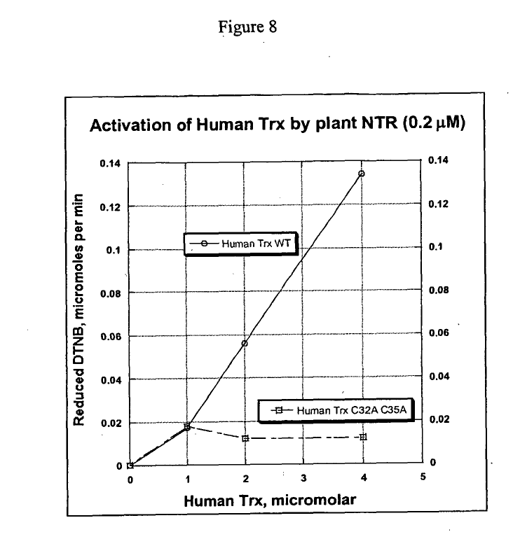

This experiment was designed to show that the C32A/C35A modified human TRX was redox inactive. The assay was run at room temperature for 15 min and the reduction of dithionitrobenzoic acid (DTNB) followed at 412 nm overtime with a spectrophotometer. The reaction mixture contains an excessive concentration of NADPH that is consumed by the TRX reductase to reduce TRX. After that, TRX reduces preferentially DTNB and TRX is recycled in its reduced form by the reductase and NADPH. FIG. 8 indicates the results obtained in this experiment, which confirms that the C32A/C35A modified human TRX is redox-inactive.

### Experiment 9

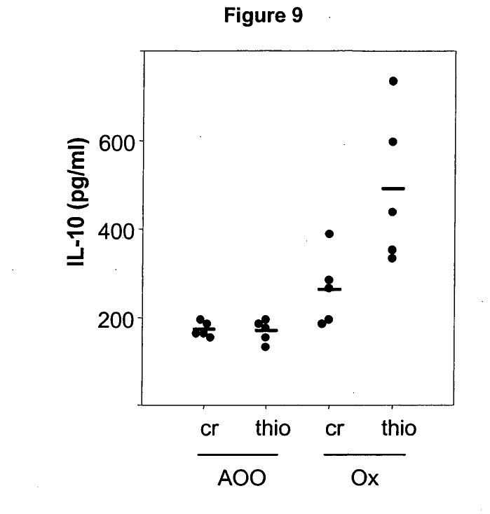

In a separate series of experiments the influence in mice of topical treatment with TRX on the elaboration by skin cells of IL-10 was measured. A representative experiment is shown in FIG. 9. Skin tissue isolated from control animals, that were exposed to vehicle (AOO) alone, but were not sensitised with the contact allergen oxazolone, produced very low levels of IL-10 and hTRX was without impact on IL-10 production. In contrast, however, hTRX was able to enhance the production of IL-10 in response to sensitisation with oxazolone. The implication is that an additional property of TRX is to augment production by skin cells of IL-10; a cytokine that is known to have anti-inflammatory effects in the skin and other tissues.

What is claimed is:

1. Use of thioredoxin in the manufacture of a medicament suitable for application to a skin surface for ameliorating an inflammatory skin condition.

2. Use according to claim 1, wherein the thioredoxin is human thioredoxin.

3. Use according to either claim 1 or claim 2, wherein the thioredoxin has the sequence depicted in SEQ ID NO. 1.

4. Use according to any one of the previous claims, wherein the thioredoxin is in a substantially reduced state.

5. Use according to any one of the previous claims, wherein the thioredoxin is in a multimeric form.

6. Use according to claim 1, wherein the thioredoxin is capable of inhibiting the production and/or activity of interleukin 1α and/or interleukin 1β.

7. Use according to claim 1, wherein the thioredoxin is capable of stimulating and/or enhancing the production and/or activity of interleukin 10.

8. Use according to claim 1, wherein the medicament is selected from the group consisting of a solution, a gel, a lotion, an ointment, a cream and a paste.

9. Use according to claim any one of the previous claims, wherein the inflammatory skin condition is selected from the group consisting of psoriasis, lichen planus, atopic eczema, irritant or allergic contact dermatitis, contact urticaria, infantile eczema and acne vulgaris.

10. Use according to claim 9, wherein the skin condition is psoriasis.

11. Use according to any one of claims 1 to 10, wherein the medicament further comprises an additional active ingredient.

12. Use according to claim 11, wherein the additional active ingredient is a corticosteroid and/or lactoferrin and/or any other topical medicament effective in the treatment of cutaneous inflammatory diseases.

13. Use according to claim 12, wherein the additional active ingredient is lactoferrin.

14. A method of ameliorating an inflammatory skin condition comprising applying to a skin surface an effective amount of a composition comprising thioredoxin.

15. A method according to claim 14, wherein said thioredoxin is human thioredoxin depicted in SEQ ID NO. 1 in a substantially reduced state.

16. A method according to claim 14 or claim 15, wherein the thioredoxin is applied to the skin surface at a concentration of 0.05 to 5 μg/cm².

17. A pharmaceutical composition suitable for ameliorating an inflammatory skin condition comprising from 0.0001 to 0.5% w/v thioredoxin.

18. A pharmaceutical composition according to claim 17, wherein the thioredoxin is human thioredoxin depicted in SEQ ID NO. 1 in a substantially reduced state.


SEQUENCE LISTING (23 sequences)

SEQ ID NO: 1 - protein, 105 aa, Homo sapiens
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV

SEQ ID NO: 2 - DNA, 315 bp, Homo sapiens
atggtgaagcagatcgagagcaagactgcttttcaggaagccttggacgctgcaggtgataaacttgtagtagttgacttctcagccacgtggtgtgggccttgcaaaatgatcaagcctttctttcattccctctctgaaaagtattccaacgtgatattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtc

SEQ ID NO: 3 - protein, 105 aa, Artificial Sequence
  Description of Artificial Sequence Synthetic polypeptide
MVKQIESKTAFQEALDAAGDKLVVVDFSATWAGPAKMIKPFFHSLSEKYSNVIFLEVDVDDCQDVASECEVKCMPTFQFFKKGQKVGEFSGANKEKLEATINELV

SEQ ID NO: 4 - DNA, 315 bp, Artificial Sequence
  Description of Artificial Sequence Synthetic polynucleotide
atggtgaagcagatcgagagcaagactgcttttcaggaagccttggacgctgcaggtgataaacttgtagtagttgacttctcagccacgtgggctgggcctgccaaaatgatcaagcctttctttcattccctctctgaaaagtattccaacgtgatattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtc

SEQ ID NO: 5 - protein, 711 aa, Homo sapiens [sequence omitted]

SEQ ID NO: 6 - DNA, 315 bp, Artificial Sequence
  Description of Artificial Sequence Synthetic polynucleotide
atggtcaaacaaattgaatctaaaaccgcattccaggaggccctggacgcggcaggggataaactggttgtggtagacttctcagcgacatggtgcggtccgtgcaaaatgatcaaaccttttttccatagtttgtccgaaaaatattcgaacgtaatcttccttgaagtcgatgtggatgactgtcaggatgtggcgagcgaatgtgaggtgaaatgcatgccaacttttcaattttttaaaaaaggccagaaagttggtgaatttagcggcgccaacaaagaaaaattagaagcgacgattaatgagctggtt

SEQ ID NO: 7 - protein, 109 aa, Escherichia coli
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 8 - protein, 114 aa, Arabidopsis thaliana
MASEEGQVIACHTVETWNEQLQKANESKTLVVVDFTASWCGPCRFIAPFFADLAKKLPNVLFLKVDTDELKSVASDWAIQAMPTFMFLKEGKILDKVVGAKKDELQSTIAKHLA

SEQ ID NO: 9 - protein, 105 aa, Artificial Sequence
  Description of Artificial Sequence Synthetic polypeptide
MVKQIESKTAFQEALDAAGDKLVVVDFSATWAGPAKMIKPFFHSLSEKYSNVIFLEVDVDDCQDVASECEVKAMPTFQFFKKGQKVGEFSGANKEKLEATINELV

SEQ ID NO: 10 - protein, 105 aa, Artificial Sequence
  Description of Artificial Sequence Synthetic polypeptide
MVKQIESKTAFQEALDAAGDKLVVVDFSATWAGPAKMIKPFFHSLSEKYSNVIFLEVDVDDAQDVASEAEVKAMPTFQFFKKGQKVGEFSGANKEKLEATINELV

SEQ ID NO: 11 - protein, 105 aa, Artificial Sequence
  Description of Artificial Sequence Synthetic polypeptide
MVKQIESKTAFQEALDAAGDKLVVVDFSATWCGPCKMIKPFFHSLSEKYSNVIFLEVDVDDCQDVASECEVKAMPTFQFFKKGQKVGEFSGANKEKLEATINELV

SEQ ID NO: 12 - protein, 105 aa, Artificial Sequence
  Description of Artificial Sequence Synthetic polypeptide
MVKQIESKTAFQEALDAAGDKLVVVDFSATWSGPCKMIKPFFHSLSEKYSNVIFLEVDVDDCQDVASECEVKCMPTFQFFKKGQKVGEFSGANKEKLEATINELV

SEQ ID NO: 13 - protein, 105 aa, Artificial Sequence
  Description of Artificial Sequence Synthetic polypeptide
MVKQIESKTAFQEALDAAGDKLVVVDFSATWCGPSKMIKPFFHSLSEKYSNVIFLEVDVDDCQDVASECEVKCMPTFQFFKKGQKVGEFSGANKEKLEATINELV

SEQ ID NO: 14 - protein, 105 aa, Artificial Sequence
  Description of Artificial Sequence Synthetic polypeptide
MVKQIESKTAFQEALDAAGDKLVVVDFSATWSGPSKMIKPFFHSLSEKYSNVIFLEVDVDDCQDVASECEVKCMPTFQFFKKGQKVGEFSGANKEKLEATINELV

SEQ ID NO: 15 - protein, 105 aa, Artificial Sequence
  Description of Artificial Sequence Synthetic polypeptide
MVKQIESKTAFQEALDAAGDKLVVVDFSATWSGPCKMIKPFFHSLSEKYSNVIFLEVDVDDCQDVASESEVKCMPTFQFFKKGQKVGEFSGANKEKLEATINELV

SEQ ID NO: 16 - protein, 105 aa, Artificial Sequence
  Description of Artificial Sequence Synthetic polypeptide
MVKQIESKTAFQEALDAAGDKLVVVDFSATWCGPSKMIKPFFHSLSEKYSNVIFLEVDVDDCQDVASESEVKCMPTFQFFKKGQKVGEFSGANKEKLEATINELV

SEQ ID NO: 17 - protein, 105 aa, Artificial Sequence
  Description of Artificial Sequence Synthetic polypeptide
MVKQIESKTAFQEALDAAGDKLVVVDFSATWCGPCKMIKPFFHSLSEKYSNVIFLEVDVDDCQDVASECEVKSMPTFQFFKKGQKVGEFSGANKEKLEATINELV

SEQ ID NO: 18 - protein, 4 aa, Artificial Sequence
  Description of Artificial Sequence Synthetic peptide
CGPC

SEQ ID NO: 19 - protein, 4 aa, Artificial Sequence
  Description of Artificial Sequence Synthetic peptide
SGPA

SEQ ID NO: 20 - protein, 4 aa, Artificial Sequence
  Description of Artificial Sequence Synthetic peptide
AGPS

SEQ ID NO: 21 - protein, 4 aa, Artificial Sequence
  Description of Artificial Sequence Synthetic peptide
AGPA

SEQ ID NO: 22 - protein, 4 aa, Artificial Sequence
  Description of Artificial Sequence Synthetic peptide
CGPA

SEQ ID NO: 23 - protein, 4 aa, Artificial Sequence
  Description of Artificial Sequence Synthetic peptide
AGPC